(19) United States
(12) Patent Application Publication
(10) Pub. No.: US 2023/0000764 A1
(43) Pub. Date: Jan. 5, 2023

(54) PRESSURIZED NUTRITIONAL SUPPLEMENT DELIVERY

(71) Applicant: BOA Nutrition, Inc., Releigh, NC (US)

(72) Inventors: Hank Durschlag, Raleigh, NC (US); Jon Pritchett, Raleigh, NC (US)

(21) Appl. No.: 17/839,128

(22) Filed: Jun. 13, 2022

Related U.S. Application Data
(60) Provisional application No. 63/209,767, filed on Jun. 11, 2021.

(51) Int. Cl. A61K 9/68; A61K 9/48; A61K 9/00

(52) U.S. Cl. CPC A61K 9/0058; A61K 9/4808; A61K 9/006

(57) ABSTRACT

Disclosed herein is a chewable article for oral mucosa delivery of a composition. The chewable system comprises a chewable base with the composition embedded, coated, integrated or dispersed therein. The composition may be pressurized, oxygenated, and/or atomized. The composition may be released as an aerosol. In one embodiment, the chewable system comprises a pressurized capsule that dissolves or ruptures in the mouth to release a burst of the composition. The chewable system may be configured to allow the composition facing to the buccal mucosa.

### CROSS REFERENCE TO RELATED APPLICATIONS

The present application claims the priority benefit of U.S. provisional patent application No. 63/208,767 filed Jun. 11, 2021, the disclosure of which is incorporated by reference herein.

### BACKGROUND OF THE INVENTION

### 1. Field of the Disclosure

The present disclosure relates to delivery of nutritional supplements through oral mucosa, and the storage and delivery devices thereof.

### 2. Description of the Related Art

Athletes may suffer excessive fluid loss through perspiration during intense exercise, which would result in electrolyte deficiency in addition to dehydration. Low levels of electrolytes in the system can lead to cramping. Salt pills provide large salt doses to overcome this issue. This approach can oversupply the body with sodium to overcome the delay in bioavailability through the GI tract. A mechanism for delivering a smaller dose of electrolytes to the buccal mucosa may provide the needed electrolytes with more rapid bioavailability.

During exercise, the body requires more oxygen than when the body is at rest. When oxygen cannot be delivered to the muscles faster to replace the used oxygen, the muscles will begin to convert the available glucose to lactic acid. This will build up in the muscle, causing fatigue. More rapid delivery of oxygen to the system during exertion can reduce fatigue. Supplemental oxygen therapy has been demonstrated to improve athletic performance and decrease reaction times.

Proper hydration, electrolyte supplements, and supplemental oxygen therapy have proven to be able to improve athletic performance. Therefore, it is desirable to have a means to delivery water, supplements and oxygen without interrupting athletes' activities.

### SUMMARY OF THE CLAIMED INVENTION

Disclosed herein is a chewable article for oral mucosa delivery of a composition. The present invention also encompasss the kit and the method of use thereof. The chewable article may comprise at least two interconnected portions. A first portion is to provide a chewable base; and a second portion is to house a composition. In one embodiment, the chewable article comprises a chewable base and a composition. The composition may be embedded, coated, integrated, encapsulated or dispersed in the chewable base. The composition may be pressurized, oxygenated, and/or atomized. The composition may be released as an aerosol. The composition may comprise fractions of supplemental nutrients, gas fraction, preservative fraction or enhancer fraction. The composition may comprise water, nutrition, or oxygen. In one embodiment, the chewable system comprises a pressurized capsule that dissolves or ruptures in the mouth to release a burst of the composition. The chewable system may be configured to allow the composition facing to the buccal mucosa.

### BRIEF DESCRIPTIONS OF THE DRAWINGS

FIG. 1 illustrates a chewable oxygenated nutritional supplement according to one embodiment of the present invention.

FIG. 2 illustrates a mouthguard according to one embodiment of the present invention.

FIG. 3 illustrates a porous bead gum, according to one embodiment of the present invention.

### DETAILED DESCRIPTION

Disclosed herein is a chewable article for oral mucosa delivery of a composition. The chewable article may comprise two portions. A first portion is to provide a chewable base; and a second portion is to hold the composition to be delivered. The first and second portions are interconnected. In one embodiment, the chewable article comprises a chewable base with the composition embedded, encapsulated, coated, integrated or dispersed therein. The composition may be pressurized, oxygenated, and/or atomized. The composition may be released as an aerosol. In one embodiment, the chewable system comprises a pressurized capsule that dissolves or ruptures in the mouth to release a burst of the composition. The chewable system may be configured to allow the composition facing to the buccal mucosa. The following embodiments/examples are included to demonstrate some specifics of the invention. It should be appreciated by those of skill in the art that the techniques disclosed in the examples that follow represent techniques discovered by the inventor to function well in the practice of the present disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the present disclosure.

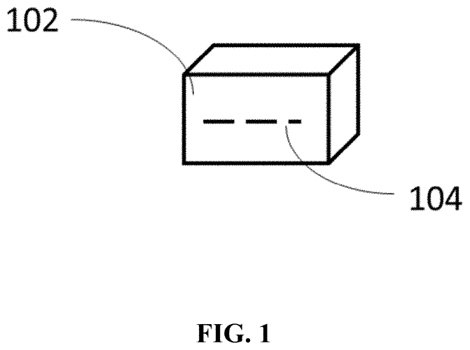

FIG. 1 illustrates one embodiment of the chewable system 102 for oral mucosa delivery of an oxygenated nutritional supplement. The chewable system 102 may comprise a chewable base with a nutritional supplement matrix embedded therein to enable transmucosal delivery of the nutritional supplement matrix. The chewable 102 may comprise any soft and/or cohesive substance suitable for chewing, such as chewing gums, gummy candies, or other food-safe substances. Chewables 102, such as gum, may contain sweeteners, flavors, colors, softeners, and/or plasticizers. The chewable 102 may comprise a chewable base with a void in the center to house the composition therein. The composition may comprise an active, such as nicotine or glucose, in the form of a liquid composition embedded inside a chewing gum. Transmucosal delivery of nutrient supplements offers advantages over oral delivery when oral delivery is impossible or negatively impacted by various issues in the gastrointestinal tract, stomach, substance digestion, absorption, swallowing, protocol compliance, substance effectiveness, or any other gastrointestinal metabolism issues.

The composition may include any one or any combinations of (i) a nutritional supplement matrix fraction, (ii) a gas fraction, (iii) an enhancer fraction, (iv) a liquid fraction, and (v) a preservative fraction. In one embodiment, the fractions are all mixed or combined and treated to allow oxygen molecules suspended therein for a specific duration of time before being released from a chewable 102. In one embodiment, a chewable 102 may contain 2 ml of the composition. Furthermore, the composition may be in a compressed state in a chewable 102 before being released. The composition may be delivered to a user via one or more perforations 104. A perforation 104 may be any intentional weak point in the chewable 102 designed to rupture when bite pressure is applied to the chewable 102, thus releasing the composition. In one embodiment, the chewable 102 has one or more perforations 104 on one side. The user may place the chewable 102 in their mouth with the perforations 104 facing their cheek. In this example, when the user applies bite pressure to the chewable 102, the composition may be released towards the buccal mucosa. In one embodiment, pressure built inside of the chewable 102 may be sufficient to spray, or otherwise atomize the composition, thus facilitating the absorption of the composition through the buccal mucosa. In one embodiment, the chewable 102 may be a gum. In one embodiment, the chewable 102 maybe a lozenge or a chewable with a hard or candy shell. In one embodiment, the chewable 102 may dissolve in the mouth without chewing. In those embodiments, the perforations 104 may dissolve faster.

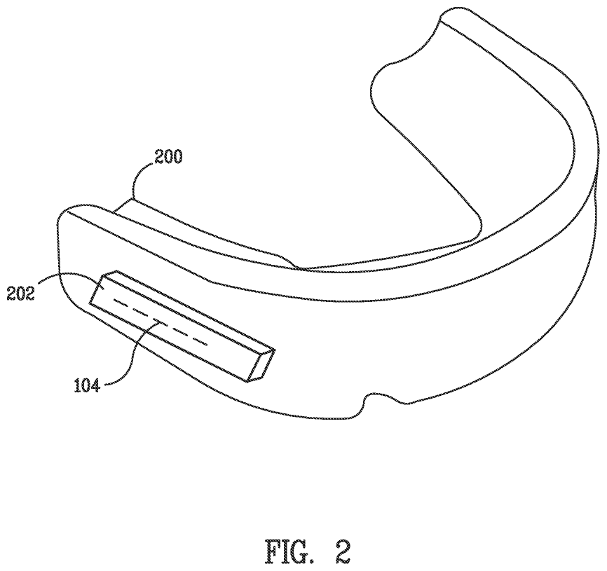

FIG. 2 illustrates one embodiment of the instant system in the form of a mouthguard 200. A mouthguard 200 may be any protective shield designed to be worn in the mouth to protect a user's teeth, lips, gums, as well as reducing the risk of oral injuries during a sport actvities. A mouthguard 200 may be customized to a user's teeth, such as made from impressions of the user's teeth, vacuum forming, bite molding, or pressure lamination. A mouthguard 200 may have a chamber 202 that houses a composition to be applied to an user's buccal mucosa. The chamber may break and releases the composition when a minimum threshold of bite pressure is applied to the mouthguard 200. The mouthguard 200 may have one or more perforations 104 positioned to release the composition from chamber 202 towards the buccal mucosa. In one embodiment, the composition may be stored in chamber 202 along with a sufficient amount of supplemental oxygen to create a burst, a blast, or an atomized spray of the composition into the user's mouth. In one embodiment, a user may wear a mouthguard 200 during an athletic event. The composition may provide the user with electrolyte supplement to sustain hydration without taking a break from the athletic activity, such as drinking from a water bottle. In one embodiment, there may be multiple chambers 202, which may be situated between the user's molars, to allow the perforations 104 adjacent to the buccal mucosa.

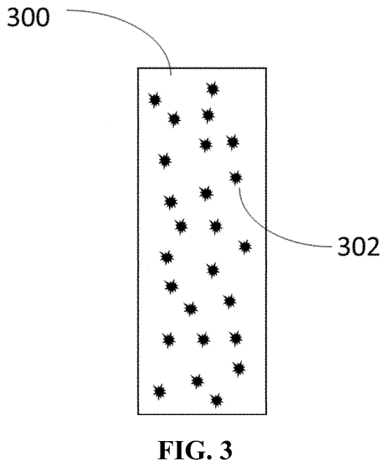

FIG. 3 illustrates another embodiment in the form of a porous bead gum 300. Gum may be impregnated with or coated with a pharmaceutical active or other biologically beneficial ingredients. Porous polymeric beads 302 may be incorporated into the gums. The porous polymeric beads 302 may act as reservoirs for housing the desired ingredient. The porous polymeric beads may quickly release the housed ingredient when the porous polymeric beads 302 are ruptured by bite pressure.

The composition, or an oxygenated portion of the composition, may be incorporated into the reservoirs and may be released from the porous bead gum 300 when chewed upon. In one embodiment, a chewable 102 may contain 2 ml of the composition in the form of 25 porous polymeric beads 302 that each contains 0.08 ml of the composition. In one embodiment, one portion of the composition may be contained in the porous polymeric beads 302. A second portion of the composition may be incorperated in the porous beads as either a coating or an ingredient. In one embodiment, the two portions of the composition may have the same ingredients with different release rates. In one embodiment, the first portion of the composition may contain a partial list of the composition's fractions. The second portion of the composition may contain the remaining fractions of the composition. For example, an electrolyte supplement may be a powder coating the porous bead gum 300, and an oxygenated flavoring may be contained in the porous polymeric beads 302. This approach may allow the user to supplement electrolytes on the go while also to receive bursts of oxygen when the porous polymeric beads 302 are ruptured.

The foregoing detailed description of the technology has been presented for purposes of illustration and description. It is not intended to be exhaustive or to limit the technology to the precise form disclosed. Many modifications and variations are possible in light of the above teaching. The described embodiments were chosen in order to best explain the principles of the technology and its practical application to thereby enable others skilled in the art to best utilize the technology in various embodiments and with various modifications as are suited to the particular use contemplated. It is intended that the scope of the technology be defined by the claim.

The terms “or” and “and/or,” as used herein, are to be interpreted as inclusive or meaning any one or any combination. Therefore, “A, B or C” or “A, B and/or C” mean any of the following: “A,” “B” or “C”; “A and B”; “A and C”; “B and C”; “A, B and C.” An exception to this definition will occur only when a combination of elements, functions, steps or acts are in some way inherently mutually exclusive.

The terms “comprising,” “including” and “having” are used interchangeably in this disclosure. The terms “comprising,” “including” and “having” mean to include, but not necessarily be limited to the things so described. The terms “about” or “approximately,” as used in the application and claims, should be understood to include the recited values or a value that is three times greater or one third of the recited values.

1. A chewable article for delivering a composition to oral mucosa, comprising;
i. a first portion for providing a chewable base, and
ii. a second portion for housing the composition;
wherein the first portion and the second portion forms an interconnection with each other; and
wherein the second portion is dissolvable or rupturable in the mouth, thus releasing the composition housed therein.

2. The chewable article of claim 1, wherein the interconnection comprises integrating, embedding, encapsulating, coating, dispersing, or any combinations thereof.

3. The chewable article of claim 1, wherein the second portion is in a form comprising a chamber, a capsule, a capsulet, a bead, a perforation, an immersion, a housing or any combinations thereof.

4. The chewable article of claim 1, wherein the composition comprises a nutrient composition, a micronutrient composition, an electrolyte composition, a nutraceutical composition, a pharmaceutical composition, a food composition, a food supplement composition, or any combinations thereof.

5. The chewable article of claim 1, wherein the composition comprises any one or any combinations of
(i) a nutritional supplement fraction;
(ii) a gas fraction;
(iii) an enhancer fraction;
(iv) a liquid fraction; or
(v) a preservative fraction.

6. The chewable article of claim 1, wherein the composition is pressurized, oxygenated, or atomized.

7. The chewable article of claim 1, wherein the composition is in a liquid form, a solid form, a gaseous form, or any combinations thereof.

8. The chewable article of claim 1, wherein the composition is released as a spray, as a burst, or as an aerosol.

9. The chewable article of claim 1, wherein the second portion is positioned to be in close proximity to buccal mucosa.

10. The chewable article of claim 1, wherein the composition is released to the oral mucosa without external force.

11. The chewable article of claim 1, wherein the composition is released to the oral mucosa through biting or chewing the chewable article.

12. The chewable article of claim 1, wherein the composition is released to the oral mucosa through applying saliva to the chewable article.

13. A kit, comprising:
i. the chewable article of claim 1;
ii. a container for housing the chewable article of claim 1; and
iii. an instruction on how to use the chewable article of claim 1.

14. A method of releasing a beneficiary composition from a chewable article, comprising:
(i) obtaining or having obtained a chewable article;
(ii) checking the integrity of the chewable article for any breakage or defects;
(iii) putting the chewable article inside the mouth if the checked chewable article is free of breakage or defects;
(iv) applying pressure and/or saliva to the chewable article at the desired moment to release the composition.

15. The method of claim 14, wherein the desired moment is before, during, or after a sport activity.

16. The method of claim 14, wherein the desired moment is during a sport activity and without interrupting the sport activity.

17. A chewable gum article comprising a gum base and beads dispersed therein, wherein the beads are rupturable by biting force.

18. The chewable gum article of claim 17, wherein the beads comprises at least one biologically beneficial ingredient.

19-20. (canceled)
